(19) United States
(12) Patent Application Publication
(10) Pub. No.: US 2011/0112326 A1
(43) Pub. Date: May 12, 2011

(54) PROCESS FOR HYDROGENATION

(76) Inventor: Jean-Paul Lange, Amsterdam (NL)

(21) Appl. No.: 12/851,462

(22) Filed: Aug. 5, 2010

(30) Foreign Application Priority Data
Aug. 7, 2009 (EP) EP09167497.8

(51) Int. Cl. C07C 51/09

(52) U.S. Cl. 562/606

(57) ABSTRACT

A process for the hydrogenation of a reactant selected from:
(a) a 5- or 6-membered lactone that is substituted at the ring-closing carbon atom and has a proton at a carbon atom adjacent to the ring-closing carbon atom;
(b) an ester of a carboxylic acid having a gamma-carbonyl group and a proton at a carbon atom adjacent to the carbon atom of the carbonyl group; and
(c) a carboxylic acid having a gamma-carbonyl group and a proton at a carbon atom adjacent to the carbon atom of the carbonyl group, which process involves contacting the reactant with a catalyst in the presence of hydrogen, at a temperature from 100 to 350° C. and a pressure from 1 to 150 bar (absolute), provided the pressure is low enough to avoid condensation of the heaviest feed component at the temperature chosen, and wherein the catalyst is a weakly acidic heterogeneous catalyst comprising a hydrogenating metal.

### FIELD OF THE INVENTION

This invention relates to processes for the catalytic hydrogenation of lactones, acids and esters of particular types, the products of which can be of use as fuel components.

### BACKGROUND TO THE INVENTION

Non-cyclic saturated esters are known to be useful as fuel components. Ethyl pentanoate is known as a gasoline component, for example, and pentyl pentanoate is a diesel component. Non-cyclic saturated esters of this type may be formed by hydrogenation of a reactant such as a lactone or a carboxylic acid or ester having a gamma-carbonyl group. Such reactants are available from biomass, in particular from cellulose feedstock material, rendering their use in the preparation of ester fuel components commercially attractive.

The catalytic hydrogenation of lactones, esters and carboxylic acids which have gamma-carbonyl groups to give ester products which are useful as fuel components is described in WO-2006/067171. In particular, the process may be used to convert gamma valerolactone (4-pentalactone or GVL) into valeric (pentanoic) acid and its esters.

The catalyst used in the process of WO-2006/067171 is a strongly acidic heterogenous catalyst comprising a hydrogenating metal on a zeolite base. By contrast, non-acidic, zeolite-free catalysts comprising a hydrogenating metal on silica, included by way of comparison in Table 1 of WO-2006/067171, are shown to give poor yields of the desired acid and ester products.

Gamma valerolactone (GVL), which may itself be prepared by catalytic hydrogenation of levulinic acid or its esters, as described in WO-2006/067171, U.S. Pat. No. 5,883,266, WO-02/074760, WO-98/26869 and EP-A-0069409, is known to be a very stable compound. As described in WO-2006/067171, GVL is more easily formed under catalytic hydrogenating conditions than non-cyclic hydrogenated compounds such as pentanoic (valeric) acid or pentanoates.

GB-1240580 describes the hydrogenolysis of esters and lactones, such as the hydrogenolysis of gamma-valerolactone to valeric acid. The hydrogenolysis of an ester or lactone to a carboxylic acid is said to be carried out in the presence of a hydrogenolysis catalyst. The hydrogenolysis catalyst is described to comprise a dual functional catalyst system or material made up of a hydrogenation component and a solid acid-acting component. The described two-component catalyst may be used as a loose physical mixture of particles of hydrogenation component and particles of acid solid or both components may be incorporated in the same particle. GB-1240580 mentions a wide range of possible solid acid-acting components. In passing acid solids of lesser acid activity, such as silica-alumina, are mentioned, but these are indicated to give lower conversions and selectivities. GB-1240580 further teaches that the greater the acid activity of the acid solid, the better conversions and selectivities.

Although GB-1240580 mentions vapor phase operation to be possible, it is indicated that generally the ester or lactone and also the product acid are in the liquid phase. GB-1240580 does not disclose a process using an acid solid of lesser acid activity, such as silica-alumina, in the vapor phase.

There remains a continuing need for improved catalysts for use in hydrogenation processes to give non-cyclic saturated carboxylic acids or esters useful as fuel components.

### SUMMARY OF THE INVENTION

The present invention provides a process for the hydrogenation of a reactant selected from:

(a) a 5- or 6-membered lactone that is substituted at the ring-closing carbon atom and has a proton at a carbon atom adjacent to the ring-closing carbon atom;

(b) an ester of a carboxylic acid having a gamma-carbonyl group and a proton at a carbon atom adjacent to the carbon atom of the carbonyl group; and

(c) a carboxylic acid having a gamma-carbonyl group and a proton at a carbon atom adjacent to the carbon atom of the carbonyl group,

comprising contacting the reactant with a catalyst in the presence of hydrogen, at a temperature from 100 to 350° C. and a pressure from 1 to 150 bar (absolute), provided the pressure is low enough to avoid condensation of the heaviest feed component at the temperature chosen, and wherein the catalyst is a weakly acidic heterogeneous catalyst comprising a hydrogenating metal.

It has now been found that, provided the pressure is low enough to avoid condensation of the heaviest feed component at the temperature chosen, weakly acidic heterogenous catalysts comprising a hydrogenating metal are slower to deactivate over prolonged use than strongly acidic catalysts and also show good selectivity for the desired non-cyclic hydrogenated compounds.

Without wishing to be bound to any kind of theory, it is thought that if a weakly acidic heterogenous catalyst, such as an amorphous silica-alumina, is used over a long period of time under liquid phase conditions, low conversions and selectivities are obtained due to leaching of part of the catalyst into the liquid phase.

It is further noted that in GB-1240580 the experiments have been run for a too short period of time to allow detection of leaching and subsequent deactivation of catalysts.

### DETAILED DESCRIPTION OF THE INVENTION

In the process of the invention, the reactant may be a lactone, a carboxylic acid having a gamma-carbonyl group and a proton at a carbon atom adjacent to the carbonyl group or an ester of such a carboxylic acid.

Where the reactant is a lactone, this is a 5- or 6-membered lactone that is substituted at the ring-closing carbon atom and has a proton at a carbon atom adjacent to the ring-closing of general molecular formula

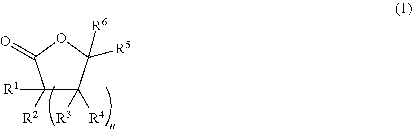

wherein n is 1 or 2, R¹, R², R³, R⁴, and R⁵ each are, independently, an proton or an organic group that is connected with a carbon atom to the carbon atom, and R⁶ is an organic group that is connected with a carbon atom to the ring-closing carbon atom. There needs to be a proton at a carbon atom adjacent to the ring-closing carbon atom. Thus, either R³ or R⁴ is a proton or any of R⁵ and R⁶ is an organic group that is connected with a proton-bearing carbon atom to the ring-closing carbon atom. In case of a 6-membered lactone, each of R³ and R⁴ at each carbon atom may differ from each other.

In one embodiment, R⁶ is an alkyl group. In another embodiment, R¹ to R⁵ each are a hydrogen atom. Examples of suitable lactones are delta hexanolactone and gamma valerolactone. In one particular embodiment, the lactone is a 5-membered lactone.

A carboxylic acid having a gamma carbonyl group and a proton (i.e. a hydrogen atom) at a carbon atom adjacent to the carbon atom of the carbonyl group or an ester thereof is suitably a compound with the general molecular formula

R⁷OOC—CR¹R²—CR³R⁴—CO—R⁶ (2)

wherein R¹, R², R³, R⁴ and R⁶ are as defined hereinabove and R⁷ is a proton in the case of a carboxylic acid as reactant and an organic group that is connected with a carbon atom to the oxygen atom in case of an ester as reactant. Suitably, R³ or R⁴ is a proton. If the carbon atom of R⁶ that is connected to the gamma carbon atom has a proton, R³ or R⁴ does not need to be a proton.

In one embodiment, the reactant is a compound that is obtainable from biomass, in particular from cellulosic or lignocellulosic material. Examples of such compounds are gamma valerolactone, levulinic acid or an ester of levulinic acid (R⁶ is a methyl group, R¹, R², R³ and R⁴ each are a H atom), a dimer of levulinic acid or a mono- or di-ester of such dimer. Examples of dimers of levulinic acid with a gamma carbonyl group are 4-methyl-6-oxononanedioic acid, 3-acetyl-4-methylheptanedioic acid, or their lactones, i.e. 5-(2-methyl-5-oxotetrahydrofuran-2-yl)-4-oxopentanoic acid or 3-(2-methyl-5-oxotetrahydrofuran-2-yl)-4-oxopentanoic acid.

The catalyst for use in the process of the present invention is a weakly acidic heterogenous catalyst comprising a hydrogenating metal. It will be appreciated that the catalyst may suitably be any weakly acidic catalytic material which is resistant to the process conditions used.

The acidity of a bifunctional catalyst may be evaluated through the heptane isomerisation test procedure as described in the examples below. The catalyst acidity is defined as the temperature that is required to achieve 40% yield in isoheptane under the conditions given below. The weaker the acidity, the higher the temperature needed for the reaction.

As used herein, a weakly acidic catalyst is a catalyst which exhibits a temperature requirement of 310-400° C. in the heptane isomerisation test procedure. Catalysts with strong acidity exhibit a temperature requirement of less than 300° C. whereas non-acidic catalysts exhibit a temperature requirement of greater than 400° C.

Hence, by a weakly acidic heterogenous catalyst comprising a hydrogenating metal is herein understood an acidic heterogeneous catalyst comprising a hydrogenating metal, which catalyst requires a temperature of 310 to 400° C. to achieve 40% yield in isoheptane in a heptanes isomerisaton test. The yield in isoheptane can suitably be quantified by means of gas chromatography.

It is well known by any skilled person in the art that an isomerisation reaction involves only rearrangement of the molecule whereby yield can be indifferently defined in terms of mole, weight or volume (when operating in gasphase as applies here). For practical purposes, especially when using gas chromatography, the yields are suitably expressed in mol %.

The present inventors have found that too weak an acid provides insufficient conversion in the hydrogenation process of the invention and that strongly acidic catalysts deactivate too quickly over prolonged periods of use. By contrast, the weakly acidic catalysts according to the present invention not only afford both acceptable activity and selectivity but they are also slower to deactivate over long periods of use. As illustrated in the examples the process of the invention can advantageously be operated during a period of at least 139 hours, preferably of at least 206 hours and most preferably of at least 334 hours. This is particularly advantageous as it reduces the frequency of catalyst regeneration and, thereby, increases its productive time. Zeolite-based strongly acidic catalysts, for example, need to be regenerated by an H₂-strip process involving heating for several hours at 400° C. under a hydrogen stream at reaction pressure without GVL feed and air-decoking for several hours at 450° C. under oxygen-lean air followed by reduction at 300° C.

In a preferred embodiment the weakly acidic heterogenous catalyst comprises a hydrogenation metal supported on a catalyst support.

The catalyst support is suitably a weakly acidic material. Preferably the catalyst support is a weakly acidic mixed oxide such as amorphous silica-alumina (ASA), Nb-, Ti- and Zr-phosphates and Ti-niobate, or a weakly acidic simple oxide such as Niobia. In one particular embodiment, the catalyst support comprises amorphous silica-alumina (ASA). The weakly acidic material may suitably be bound with a binder, for example silica, alumina, acidic clays, titania or zirconia.

Although weakly acidic zeolite bases may be envisaged, in one embodiment, the weakly acidic heterogenous catalyst comprises a zeolite free catalyst.

The hydrogenating metal of the catalyst suitably comprises a metal of any one of groups 7 to 11 of the Periodic Table of Elements such as Ni, Rh, Pd, Pt, Re, Ru or a combination of two or more thereof.

In one embodiment, the hydrogenating metal comprises Pt, Pd or a combination thereof, optionally additionally with one or more other metals from groups 7-11 of the Periodic Table of Elements. In one particular embodiment, the hydrogenating metal comprises both Pt and Pd.

The concentration of the hydrogenating metal based on the total weight of the catalyst will typically be in the range of from 0.05 to 5 wt %, suitably from 0.1 to 2 wt %.

In another particular embodiment, the catalyst comprises hydrogenating metal supported on the weakly acidic material.

The process of the invention is conveniently conducted at a temperature in the range of 150-350° C., particularly 200-300° C., more particularly 250-300° C. It will be appreciated that the temperature may be varied depending on the metals present in the catalyst and the support used.

The process of the invention may be performed at any suitable pressure provided that it is low enough to avoid condensation of the heaviest feed component at the temperature chosen. This is understood to mean that the process of the invention is carried out under gasphase conditions.

The reactant is suitably contacted with the catalyst at a pressure of 1-150 bar. In one embodiment, the process is conducted at a pressure of 5-50 bar.

The products of the process of the present invention are non-cyclic, saturated carboxylic acids and esters. An ester will be formed, for example, where the feedstock is itself an ester or if an alcohol is added to the feedstock; alternatively, where the product is a carboxylic acid, this can be esterified subsequently to give an ester.

The ester products can be of use as fuel components, for example in transportation fuels, for example as gasoline or diesel fuels. Suitable esters include those formed by reacting gamma valerolactone, levulinic acid, or its esters or by reacting dimers of levulinic acid, such as 4-methyl-6-oxononanedioic acid, 3-acetyl-4-methylheptanedioic acid, their esters, or their lactones. The resulting esters are in that case esters of pentanoic acid (gamma valerolactone, levulinic acid or its esters as reactant), di-esters of 4-methylnonanedioic acid (4-methyl-6-oxononanedioic acid, its lactone, or its (di)ester as reactant) or di-esters of 3-ethyl-4-methylheptanedioic (3-acetyl-4-ethylheptanedioic acid, its lactone, or its (di)ester as reactant). The ethyl esters are particularly preferred as fuel components.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

### EXAMPLES

The invention will now be further illustrated by means of the following non-limiting examples.

### Example 1

### Evaluation of Catalyst Acidity in the Heptane Isomerisation Test Procedure

A known amount (0.35 g) of catalyst was loaded in a reactor and reduced for 1.5 h at 440° C. under H₂ flow of gas hourly space velocity (GHSV) 6200 L/kg/h at 30 bar. The catalyst was subsequently contacted with a flow of n-heptane in H₂ (4 vol %) at GHSV of 4000 L/kg/h and 30 bar and cooled down to 200° C. over 20 hours while the yield of isoheptane was quantified by means of gas chromatography.

The temperature required to achieve 40% yield of isoheptane for various weakly acidic catalysts was determined and the results are presented in Table 1 below. Results are also presented for three strongly acidic zeolite catalysts and two non-acidic catalysts by way of comparison. Catalysts were prepared according to the methods described in Example 2 below.

ASA supports were obtained commercially from CRI (X-600) and Catalysts & Chemicals Industrial Co. Ltd (CCIC).

H-ZSM-5, H-ZSM-12 and H-Beta are commercially available zeolites.

Silica (SiO₂) and alumina (Al₂O₃) were obtained commercially from CRI.

The supports were impregnated by Pt and/or Pd by means of well-known incipient wetness impregnation.

TABLE 1

| | | T required |
|---|---|---|
| | Metal | [° C. |
| Support | | |
| X-600 ASA | 0.3% Pt + 1% Pd | 379 |
| X-600 ASA | 0.8% Pt | 360 |
| CClC ASA | 0.8% Pt | 347 |
| CClC ASA | 0.3% Pt + 0.5% Pd | 340 |
| Reference Materials | | |
| H-ZSM-5/SiO₂ | 0.7 Pt | 273 |
| H-ZSM-12/SiO₂ | 0.7 Pt | 302 |
| H-Beta/SiO₂ | 1% Pt | 292 |
| SiO₂ | 0.8% Pt | 500 |
| γ-Al₂O₃ | 0.8% Pt | 482 |

The catalyst acidity is defined as the temperature that is required to achieve 40% yield in isoheptane. Catalysts with strong, weak and no acidity exhibit a temperature requirement of <300° C., 310-400° C. and >400° C., respectively.

Table 1 shows clearly that catalysts based on ASA are ‘weakly acidic’. In contrast, catalysts based on ZSM-5, ZSM-12 and Beta zeolites are ‘strongly acidic’ whereas catalysts based on SiO₂ and Al₂O₃ or ‘non-acidic’.

### Example 2

### Catalyst Preparation and Hydrogenation Process

Various catalysts of the invention having a hydrogenating metal supported on a weakly acidic support and reference catalysts supported on either strongly acidic or non-acidic supports were prepared using an incipient wetness impregnation procedure. Before impregnation, the supports were pre-dried at 300° C. for 1 hour, The required amount of metal solution was calculated and prepared based on the pore volumes of the supports and the desired metal loading such that the total volume of the solution for impregnation was enough to fill 95%+/−5% of the support pores.

Gammavalerolactone (GVL) was catalytically reduced using a process according to the present invention. The experiments were carried out using either a four-barrel microflow unit that was equipped with Hastelloy HC 276 reactor (1 cm ID) or in a 16-barrel unit using SS316 reactors. 5 g catalysts were loaded in the reactors as trilobes (1.6 mm diam.) as 5 batches of 1 g each that were separated with a charge of 1.4 SiC (0.2 mm). The catalysts were reduced for 3 h at 300° C. under a H₂ flow of 15 NL/h at atmospheric pressure. The reactors were then cooled to 250° C. and fed with a pure GVL feed (from Innochem) and H₂ under the conditions specified in Tables 2 and 3 below.

The % conversion of the GVL was monitored, providing an indication of the stability of catalyst activity. Also monitored was the percentage of the desired reaction product valeric acid, and various by-products, as an indicator of catalyst selectivity. The stability and selectivity results are summarised in Tables 2 and 3.

TABLE 2
Long-term experiments to compare the activity and selectivity
of noble metals supported on amorphous silica alumina (ASA),
zeolites (ZSM-5, MOR, MWW) and W/ZrO₂.

| | | GVL | Va | |
|---|---|---|---|---|
| | | Conver- | selec- | |
| | t | sion | tivity | |
| Catalyst | [h] | [%] | [%] | Conditions |
| 0.3% Pt + 0.5% | 10 | 77.0 | 83 | 250° C., 10 bar, |
| Pd/X-600 ASA | | | | 2 g/g/h, FR = 9 |
| | 25 | 79.3 | 87 | |
| | 55 | 79.2 | 93 | |
| | 103 | 79.2 | 89 | |
| | 139 | 78.1 | 91 | |
| 0.8% Pt/X-600 | 6 | 73.5 | 91 | 250° C., 10 bar, |
| | | | | 2 g/g/h, FR = 9 |
| | 25 | 74.9 | 77 | |
| | 55 | 78.1 | 68 | |
| | 103 | 71.5 | 81 | |
| | 139 | 69.3 | 85 | |
| 0.8% Pt/CClC | 6 | 78.6 | 85 | 250° C., 10 bar, |
| ASA | | | | 2 g/g/h, FR = 9 |
| | 25 | 73.4 | 89 | |
| | 67 | 69.6 | 95 | |
| | 103 | 70.0 | 89 | |
| | 139 | 66.7 | 98 | |
| 0.3% Pt + 0.5% | 10 | 86.1 | 72 | 250° C., 10 bar, |
| Pd/IC/ClC ASA | | | | 2 g/g/h, FR = 9 |
| | 25 | 84.1 | 81 | |
| | 55 | 81.5 | 84 | |
| | 103 | 79.4 | 85 | |
| | 139 | 77.6 | 88 | |
| 0.8% Pt/HZSM5/ | 6 | 79 | 89 | 250° C., 10 bar, |
| SiO₂ | | | | 6.9 g/g/h, FR = 10 |
| | 22 | 45 | 94 | |
| | 54 | 29 | 83 | |
| | 106 | 21 | 78 | |
| | 206 | 18 | 70 | |
| | 334 | 12 | 76 | |
| 0.1% Pd/W/Zr | 6 | 67 | 85 | 250° C., 10 bar, |
| (12) | | | | 2.8 g/g/h, FR = 10 |
| | 22 | 51 | 91 | |
| | 54 | 38 | 84 | |
| | 106 | 34 | 79 | |
| | 206 | 24 | 74 | |
| | 334 | 20 | 62 | |
| 0.3% Pt/H-MOR/ | 6 | 78 | 81 | 250° C., 10 bar, |
| SiO₂ | | | | 2 g/g/h, FR = 10 |
| | 22 | 69 | 81 | |
| | 54 | 54 | 91 | |
| | 106 | 45 | 90 | |
| | 206 | 34 | 92 | |
| | 334 | 28 | 85 | |
| 0.3% Pt/H-MWW/ | 6 | 79 | 87 | 250° C., 10 bar, |
| SiO₂ | | | | 2 g/g/h, FR = 10 |
| | 22 | 55 | 88 | |
| | 54 | 41 | 82 | |
| | 106 | 36 | 68 | |
| | 206 | 26 | 77 | |
| | 334 | 23 | 69 | |

TABLE 3
Screening experiments with one ASA and four mixed
oxides compared to one zeolite (ZSM-5) and W/ZrO₂.

| | | GVL | VA | |
|---|---|---|---|---|
| | | conversion | selectivity | |
| Catalyst | t [h] | [%] | [%] | Conditions |
| 0.3% Pt 0.5% | 5 | 66 | 72 | 250° C., 10 bar, |
| Pd/ASA | | | | 2 g/g/h, FR = 10 |
| | 24 | 61 | 77 | 250° C., 10 bar, |
| | | | | 2 g/g/h, FR = 10 |
| 1% Pt/NbPO4 | 5 | 22 | 68 | 250° C., 10 bar, |
| | | | | 2 g/g/h, FR = 10 |
| | 24 | 16 | 58 | 250° C., 10 bar, |
| | | | | 2 g/g/h, FR = 10 |
| 1% Pt/TiPO4 | 5 | 14 | 94 | 250° C., 10 bar, |
| | | | | 2 g/g/h, FR = 10 |
| | 24 | 26 | 65 | 250° C., 10 bar, |
| | | | | 2 g/g/h, FR = 10 |
| 1% Pt/ZrPO4 | 5 | 17 | 68 | 250° C., 10 bar, |
| | | | | 2 g/g/h, FR = 10 |
| | 24 | 50 | 60 | 250° C., 10 bar, |
| | | | | 2 g/g/h, FR = 10 |
| 1% Pt/TiNbO5 | 5 | 18 | 64 | 250° C., 10 bar, |
| | | | | 2 g/g/h, FR = 10 |
| | 24 | 19 | 61 | 250° C., 10 bar, |
| | | | | 2 g/g/h, FR = 10 |
| 0.8% Pt/H-ZSM- | 5 | 93 | 90 | 250° C., 10 bar, |
| 5/SiO₂ | | | | 5 g/g/h, FR = 10 |
| | 24 | 30 | 70 | 250° C., 10 bar, |
| | | | | 5 g/g/h, FR = 10 |
| 0.3%/Pd/W/Zr | 5 | 77 | 76 | 250° C., 10 bar, |
| (8) | | | | 2 g/g/h, FR = 10 |
| | 24 | 59 | 86 | 250° C., 10 bar, |
| | | | | 2 g/g/h, FR = 10 |

In Tables 2 and 3, FR is feed ratio (H₂/GVL in mol/mol).

Catalysts are defined in terms of the hydrogenation metal/acidic function/inert binder (where present), with the metal loading given in weight %. In the catalysts used, the acidic function is a zeolite (H-ZSM-5, H-MWW, H-MOR using the nomenclature published in the atlas of zeolite structure types “W. M. Meier, D. H. Olson, Ch. Baerlocher, Zeolites 1996, 17, 1-230”), an amorphous silica-alumina (ASA), tungsten oxide supported on zirconia (W/Zr), metal phosphates (NbPO₄, TiPO₄, ZrPO₄) or Titanium niobate (TiNbO₅). The inert binder is SiO₂.

From the results presented in Tables 2 and 3 above it can be seen that, the weakly acidic catalysts of the present invention not only show good activity and selectivity but they are significantly more stable to deactivation than are the strongly acidic zeolite and W/ZrO₂ based catalysts. The weakly acidic ASA based catalysts in particular show exceptional stability over a long run of one week, with the PtPd catalysts being slightly more active than their Pt counterparts. Although the weakly acidic catalysts supported on Nb-, Ti- and Zr-phosphates and Ti-niobate show modest activity, it can be seen from the results presented in Table 3 that these retain their activity and selectivity after one day in contrast to the strong acid based catalysts which undergo deactivation under the same conditions. The Ti- and ZrPO₄ catalysts even gained in activity over the initial 24 h.

### Comparative Example 3

To illustrate the effects of liquid phase conditions on a weakly acidic heterogeneous catalyst, amorphous silica-alumina was subjected to a leaching test involving cooking 1 g of amorphous silica-alumina (ASA) shaped as extrudates for approximately one week in 10 g of liquid levulinic acid at 150° C. Following this test, integrity of the amorphous silica-alumina was inspected visually and an element analysis of the liquid phase was performed to establish whether there had been material leaching. Results are presented in Table 4 below.

TABLE 4

| Material | [Si/ppm] | [M/ppm] | M |
|---|---|---|---|
| ASA (X600, commercially | catalyst dissolved | | |
| obtained from CRI) | | | |
| ASA (MS13/110W ASA | <0.5 | 3245 | Al |
| commercially obtained | | | |
| from Grace Davison) | | | |

What is claimed is:

1. A process for the hydrogenation of a reactant selected from the group consisting of:
(a) a 5- or 6-membered lactone that is substituted at the ring-closing carbon atom and has a proton at a carbon atom adjacent to the ring-closing carbon atom;
(b) an ester of a carboxylic acid having a gamma-carbonyl group and a proton at a carbon atom adjacent to the carbon atom of the carbonyl group; and
(c) a carboxylic acid having a gamma-carbonyl group and a proton at a carbon atom adjacent to the carbon atom of the carbonyl group,
Comprising contacting the reactant with a catalyst in the presence of hydrogen, at a temperature from 100 to 350° C. and a pressure from 1 to 150 bar (absolute), provided the pressure is low enough to avoid condensation of the heaviest feed component at the temperature chosen, and wherein the catalyst is a weakly acidic heterogeneous catalyst comprising a hydrogenating metal.

2. The process of claim 1 wherein the reactant is a 5- or 6-membered lactone that is substituted at the ring-closing carbon atom and has a proton at a carbon atom adjacent to the ring-closing carbon atom.

3. The process of claim 1 wherein the reactant is gamma-valerolactone.

4. The process of claim 1 wherein the reactant is contacted with the catalyst at a temperature of from 200° C. to 350° C.

5. The process of claim 1 wherein the reactant is contacted with the catalyst at a pressure of from 5-50 bar (absolute).

6. The process of claim 1 wherein the hydrogenating metal comprises a metal of any one of groups 7 to 11 of the Periodic Table of Elements or a combination of two or more such metals.

7. The process of claim 6 wherein the hydrogenating metal comprises platinum, palladium or a combination thereof.

8. The process of claim 1 wherein the weakly acidic catalyst comprises a weakly acidic mixed oxide or a weakly acidic simple oxide.

9. The process of claim 1 wherein the catalyst comprises a zeolite-free catalyst.

10. The process of claim 1 wherein the catalyst comprises amorphous silica-alumina, niobium phosphate, titanium phosphate, zirconium phosphate, titanium niobate or niobium oxide.

11. The process of claim 10 wherein the catalyst comprises amorphous silica-alumina.

12. The process of claim 1 wherein the catalyst comprises hydrogenating metal supported on a weakly acidic material.

13. The process of claim 1 wherein the catalyst comprises platinum and palladium supported on an amorphous silica-alumina.

14. The process of claim 1 wherein the process is operated during a period of at least 334 hours.

15. The process of claim 2 wherein the reactant is contacted with the catalyst at a temperature of from 200° C. to 350° C.

16. The process of claim 15 wherein the reactant is contacted with the catalyst at a pressure of from 5-50 bar (absolute).

17. The process of claim 2 wherein the catalyst comprises amorphous silica-alumina, niobium phosphate, titanium phosphate, zirconium phosphate, titanium niobate or niobium oxide.

18. The process of claim 17 wherein the reactant is contacted with the catalyst at a temperature of from 200° C. to 350° C.

19. The process of claim 18 wherein the reactant is contacted with the catalyst at a pressure of from 5-50 bar (absolute).
